(19) United States
(12) Patent Application Publication
(10) Pub. No.: US 2026/0248485 A1
(43) Pub. Date: Aug. 27, 2026

(54) SYSTEM FOR ANGLE ESTIMATION AND ADJUSTMENT FOR BI-PLANE ULTRASOUND IMAGING

(71) Applicant: GE Precision Healthcare LLC, Waukesha, WI (US)

(72) Inventors: Ella Sokulin, Kiryat Tivon (IL); Alexander Sokulin, Kiryat Tivon (IL); Doron Shaked, Kiryat Tivon (IL); Carmit Shiran, Middleton, WI (US); Roei Gelbhart, Tel-Aviv (IL); Menachem Halmann, Monona, WI (US); Or Elezra, Haifa (IL); Orit Dudek, Haifa (IL); Shreya Bhise, Milwaukee, WI (US)

(73) Assignee: GE Precision Healthcare LLC, Waukesha, WI (US)

(21) Appl. No.: 19/063,132

(22) Filed: Feb. 25, 2025

(51) Int. Cl. A61B 8/00; G06V 10/26; G06V 10/764

(52) U.S. Cl. CPC A61B 8/42; A61B 8/463; A61B 8/483; G06V 10/26; G06V 10/764; G06V 2201/03

(57) ABSTRACT

An ultrasound system may include a transducer configured to transmit and receive an ultrasound signal in a first plane and a second plane. The system may include a processing circuit to perform operations. The operations include receiving a first image data obtained by an ultrasound probe along the first plane, receiving a second image data obtained by the ultrasound probe along the second plane, and identifying an anatomical feature based on the first image data and the second image data. The operations include determining an angle between an ultrasound beam and a normal of the anatomical feature, determining a transmit direction that would adjust the angle, and at least one of displaying the transmit direction on a user interface of the ultrasound imaging system or automatically aligning a second ultrasound signal in the transmit direction to adjust the angle to the desired angle.

### STATEMENT REGARDING FEDERALLY SPONSORED RESEARCH AND DEVELOPMENT

This invention was made with government support under Grant No. 75A50123C00035 awarded by Biomedical Advanced Research and Development Authority (BARDA). The Government has certain rights in the invention.

### FIELD

Embodiments of the subject matter disclosed herein relate to ultrasound imaging, and more particularly, to the use of bi-plane ultrasound imaging for angle estimation and adjustment of an ultrasound beam to an anatomical feature.

### BACKGROUND

During an ultrasound scan of a lung, an ultrasound probe is placed in a first orientation (e.g., a sagittal orientation towards a patient's head) by a technician, such as a sonographer. Identifying a pleura of the lung is a challenge during the scan and the technician may move the ultrasound probe from the first orientation to a second orientation to capture a better scan of the pleura which occurs when an ultrasound beam from the ultrasound probe is perpendicular to the pleura. Images obtained during the ultrasound scan of the lung may depict artifacts, false positive diagnosis of pleural irregularity, loss of pleura brightness, and A-line artifacts if image quality is not optimal.

### SUMMARY

An embodiment relates to an ultrasound imaging system. The ultrasound imaging system includes a transducer configured to transmit and receive an ultrasound signal in a first plane and a second plane, with the first plane orthogonal to the second plane. The transducer emits an ultrasound beam. The ultrasound imaging system also includes a processing circuit. The processing circuit includes a processor coupled to a memory device storing instructions thereon that, when executed, cause the processing circuit to perform operations including receiving a first image data obtained by an ultrasound probe along the first plane, receiving a second image data obtained by the ultrasound probe along the second plane, identifying an anatomical feature based on the first image data and the second image data, determining, based on the first image data and the second image data, an angle between the ultrasound beam and a normal of the anatomical feature, determining a transmit direction that would adjust the angle between the ultrasound beam and the normal of the anatomical feature to a desired angle, and at least one of displaying the transmit direction on a user interface of the ultrasound imaging system or automatically aligning a second ultrasound signal in the transmit direction to adjust the angle to the desired angle.

Another embodiment relates to an ultrasound imaging system. The ultrasound imaging system includes a transducer configured to transmit and receive an ultrasound signal in a first plane and a second plane, with the first plane orthogonal to the second plane. The transducer emits an ultrasound beam, and the transducer is used to obtain first image data along the first plane and the transducer is used to obtain second image data along the second plane. The ultrasound imaging system includes an image processing circuit configured to identify an anatomical feature based on the first image data and the second image data, where an angle between the ultrasound beam and a normal of the anatomical feature is determined. The ultrasound imaging system also includes a control circuit configured to compute a transmit direction that would adjust the angle between the ultrasound beam and the normal of the anatomical feature towards a desired angle, such that the ultrasound imaging system is configured to at least one of display the transmit direction on a user interface of the ultrasound imaging system or automatically align a second ultrasound signal in the transmit direction to adjust the angle to the desired angle.

Another embodiment relates to a method. The method includes receiving, by a processing circuit, a first image data obtained by an ultrasound probe along a first plane, the ultrasound probe configured to transmit an ultrasound beam. The method includes receiving, by the processing circuit, a second image data obtained by the ultrasound probe along a second plane, where the second plane is orthogonal to the first plane. The method also includes identifying, by the processing circuit, an anatomical feature based on the first image data and the second image data. The method also includes determining, by the processing circuit, the first image data, the second image data, an angle between the ultrasound beam, and a normal of the anatomical feature. The method also includes determining, by the processing circuit, a transmit direction that would adjust the angle between the ultrasound beam and the normal of the anatomical feature towards a desired angle. The method includes at least one of displaying the transmit direction on a user interface or automatically aligning a second ultrasound signal in the transmit direction to adjust the angle to the desired angle.

This summary is illustrative only and is not intended to be in any way limiting. Other aspects, inventive features, and advantages of the devices or processes described herein will become apparent in the detailed description set forth herein, taken in conjunction with the accompanying figures, wherein like reference numerals refer to like elements.

### BRIEF DESCRIPTION OF THE DRAWINGS

FIG. 1 is a block diagram of an ultrasound imaging system, according to an example embodiment.

FIG. 2 is a diagram of a desired orientation of an ultrasound probe used in the ultrasound imaging system of FIG. 1, according to an example embodiment.

FIG. 3 is an illustration of a configuration of the ultrasound probe used in the ultrasound imaging system of FIG. 1, according to an example embodiment.

FIG. 4 is a flow chart illustrating a method for bi-plane ultrasound imaging using the ultrasound imaging system of FIG. 1, according to an example embodiment.

FIG. 5 is a flow chart illustrating a method for angle estimation and ultrasound probe adjustment during the method of FIG. 4, according to an example embodiment.

FIG. 6A is an illustration of a first ultrasound image acquired using a first type of ultrasound probe, according to an example embodiment.

FIG. 6B is an illustration of a second ultrasound image acquired using the first type of ultrasound probe, according to an example embodiment.

FIG. 7A is an illustration of the first ultrasound image of FIG. 6A after processing, according to an example embodiment.

FIG. 7B is an illustration of the second ultrasound image of FIG. 6B after processing, according to an example embodiment.

FIG. 8A is an illustration of a first ultrasound image acquired using the first type of ultrasound imaging, according to an example embodiment.

FIG. 8B is an illustration of a second ultrasound image acquired using the first type of ultrasound imaging, according to an example embodiment.

FIG. 9 is an illustration of the first ultrasound image of FIG. 6A after processing, according to an example embodiment.

FIG. 10A is an illustration of a first ultrasound image acquired using a first type of ultrasound imaging after segmentation, according to an example embodiment.

FIG. 10B is an illustration of the first ultrasound image of 10A after a vector determination, according to an example embodiment.

### DETAILED DESCRIPTION

Referring generally to the figures, systems and methods for the use of bi-plane ultrasound imaging for angle estimation and adjustment of an ultrasound beam to an anatomical feature. The systems disclosed herein are used to determine an angle between an ultrasound beam and a normal of an anatomical feature to determine a transmit direction to shift the angle towards a desired angle. The systems disclosed herein use a first plane and a second plane that are orthogonal to each other (e.g., an azimuth plane and an elevation plane) to determine the angle and the transmit direction. The systems and methods use the transmit direction such that a user interface can display the transmit direction and/or a probe and/or ultrasound beams of the system can be automatically adjusted to the transmit direction.

During an ultrasound scan of a lung, an ultrasound probe is conventionally positioned in a sagittal orientation (e.g., pointed towards a patient's head). Generally, however, identifying a pleura of the lung is challenging and an acquired image at the orientation may not be of proper quality. Visualization of pleura is important for evaluating the lung and searching for pathologies around the pleura. The lack of quality in the image may be due to artifacts, a false positive diagnosis of pleural irregularity, a lack of visualization of A-lines, and a loss of pleura brightness. Thus, it is desired to orient the probe such that the ultrasound beam from the probe is perpendicular to the pleura. At such an orientation, the pleura is at an optimal view and appears as a well-defined echogenic line. Manually orienting the ultrasound probe without assistance such that the ultrasound beam is perpendicular to the pleura is time consuming and relies on the skills/expertise of the operator (e.g., sonographer, technician, clinician, etc.), thereby resulting in inconsistencies in operator efficiencies and in obtaining consistent and high-quality ultrasound images.

In some cases, the operator can use visualization of the A-lines as an indication of good angular position of the pleura; however, when the pleura is not at the optimal view, the A-lines may not be visualized well and positioning the probe such that the ultrasound beam is perpendicular to the pleura without visualization of the A-lines requires additional precision, skill, and expertise of the operator. Additionally, both unhealthy and healthy patients may have A-lines that cannot be visualized, so reliance on A-lines is not always an option for the operator. The variance of A-lines visualization between patients means the operator's skills/expertise relating to probe navigation also rely on knowledge of the specific anatomy of the patient being imaged, which the operator typically lacks.

The systems and methods described herein provide a technical solution to existing ultrasound imaging systems by implementing a probe configured to capture ultrasound data from two planes and use an algorithm to determine a current angle of the ultrasound beam and a normal of an anatomical feature and a transmit direction of the probe that would adjust the current angle towards a desired angle based on an assessment of a patient's anatomy. Therefore, a second ultrasound signal may be automatically aligned in the adjusted transmit direction or the adjusted transmit direction can be displayed for the operator, as described herein, such that the operator can manually adjust the probe. Furthermore, using the systems and methods described herein, real-time feedback can be provided to the operator to aid in moving the probe to a desired orientation for scanning.

Thus, the systems and methods described herein reduce the dependency on the expertise and skills of the operator by automatically determining a transmit direction of the probe for achieving a desirable angle between the ultrasound beam and the anatomical feature. Additionally, the system may automatically adjust the probe (e.g., with adjustment of a transmitter beamform) to the transmit direction, thereby reducing the need for the operator to maneuver the probe manually to reach that view. Furthermore, the systems and methods described herein assist an operator in detecting a pathology and/or completing an ultrasound exam in a shorter amount of time (e.g., due to the operator not having to manually adjust the probe to obtain the desired transmit direction).

The implementations described herein address a technical problem by providing enhanced data integration and analysis capabilities, which deliver a particular technical solution that streamlines and refines generating bi-plane images during a lung ultrasound. The systems described herein are implemented to improve how data is synthesized and utilized from various sources that provide information relating to an optimal adjustment of a probe for capturing images during an ultrasound scan. By assessing specific anatomical features and automatically adjusting a transmit direction of the probe based on the assessment, these systems provide real-time guidance for generating bi-plane images during an ultrasound scan. Accordingly, this approach provides a specific technical improvement to various technical problems, including those set forth herein. The systems described herein may also reduce processing power by performing various processing operations simultaneously to generate bi-plane images during an ultrasound scan, rather than performing a plurality of processing operations individually and consuming unnecessary processing power.

Before turning to the figures, which illustrate certain exemplary embodiments in detail, it should be understood that the present disclosure is not limited to the details or methodology set forth in the description or illustrated in the figures. It should also be understood that the terminology used herein is for the purpose of description only and should not be regarded as limiting.

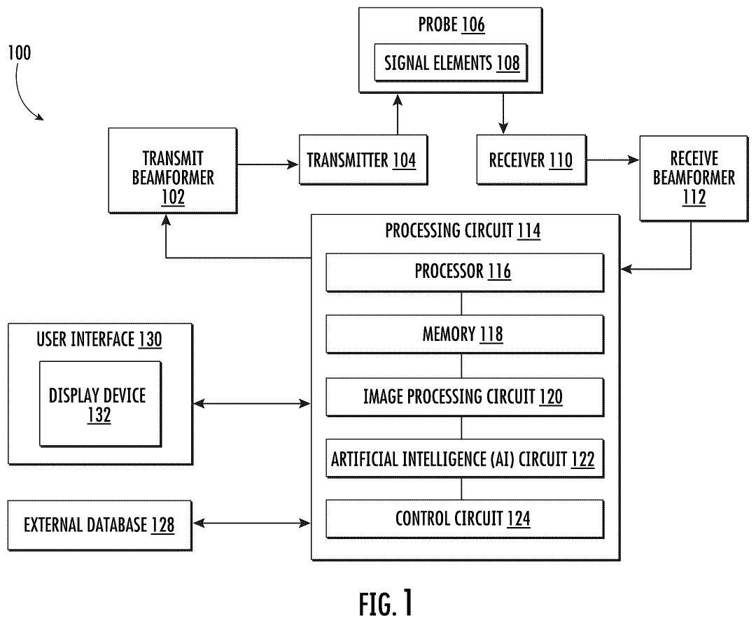

Referring to FIG. 1, a schematic diagram of an ultrasound imaging system 100 is shown. The ultrasound imaging system 100 may be used in a medical environment (e.g., hospitals, clinics, etc.), for example, by a sonographer, technician, or other clinician certified to collect ultrasound data from a patient.

An example of a procedure performed using the ultrasound imaging system 100 may be a lung ultrasound. The lung ultrasound may be performed to detect various pulmonary pathologies such as pneumonia, pulmonary edema, pleural effusion, pneumothorax (e.g., a collapsed lung), pulmonary embolism, lung cancer, and so on. Such pathologies are detected by collecting and processing ultrasound data (e.g., using the ultrasound imaging system 100, as described herein). During the lung ultrasound, a sonographer collects the ultrasound data by navigating a probe (e.g., probe 106, as described below) over a patient's chest until a sufficient volume of ultrasound images are collected. The sonographer may collect the ultrasound images both with the ultrasound probe in a sagittal orientation and with the ultrasound probe in a transverse orientation. For example, the transverse orientation may be particularly beneficial in instances where a pulmonary pathology is detected. The collected images are stored in a central storage device (e.g., memory 118) and analyzed by the sonographer. The sonographer generates a set of measurements from the images (e.g., 50-100 records), and the images and measurements are collectively reviewed by a medical expert, such as a pulmonologist. The pulmonologist provides any clinical findings/conclusions in a report submitted to the patient's medical record.

As shown in FIG. 1, the ultrasound imaging system 100 includes a transmit beamformer 102, a transmitter 104, a probe 106, a receiver 110, and a receive beamformer 112. The ultrasound imaging system 100 may also include a matching layer and a damping block. The matching layer improves ultrasound image quality by having an acoustic impedance between a tissue to be imaged and a material of a transducer of the ultrasound imaging system 100. The damping block is configured to absorb ultrasound energy to improve ultrasound image quality.

The transmit beamformer 102 may be either a hardware beamformer or a software beamformer. In embodiments where the transmit beamformer 102 is a hardware beamformer, the transmit beamformer 102 may include one or more of a graphics processing unit (GPU), a microprocessor, a central processing unit (CPU), a digital signal processor (DSP), or any other type of processor capable of performing logical operations. The transmit beamformer 102 may be configured to perform conventional beamforming techniques as well as techniques such as retrospective transmit beamforming (RTB). Alternatively, in embodiments where the transmit beamformer 102 is a software beamformer, a processor (e.g., processor 116, as described below) may be configured to perform some or all of the functions associated with the transmit beamformer 102.

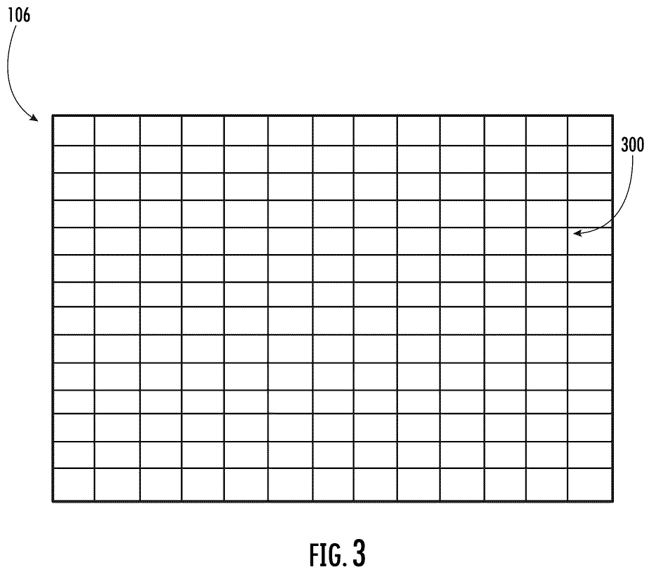

The probe 106 may be a linear array probe, a curvilinear array probe, a sector probe, or any other type of probe configured to obtain ultrasound data (e.g., B-mode data, color flow data, etc.). More specifically, the probe 106 may be any type of probe including a matrix transducer array (e.g., matrix configuration 300, as described below with reference to FIG. 3) configured to obtain three-dimensional (3D) ultrasound data. In some embodiments, the probe 106 may include a position sensor configured to detect a position of the probe 106 relative to one or more reference locations. That is, the position sensor may continuously track movement (e.g., rotation, translation, orientation, etc.) of the probe 106 relative to the location of the probe 106 when the anatomy being imaged is identified. For example, the anatomy being imaged may be identified as a pleura (e.g., represented by pleura 602, as described below) at a first location of the probe 106. Then, the position sensor may track the movement of the probe 106 relative to the pleura to identify successive locations of the probe 106. In some embodiments, the position sensor may transmit position data to be stored within the ultrasound imaging system 100 (e.g., in memory 118).

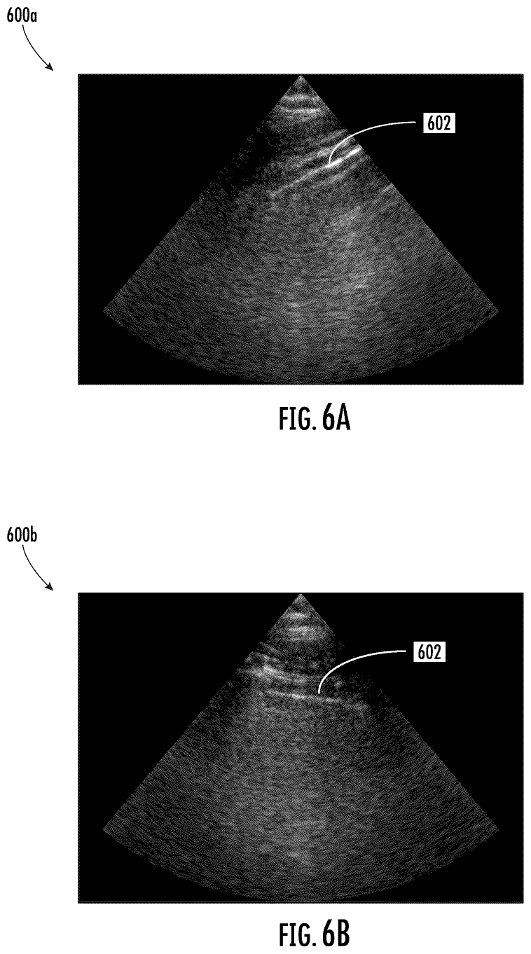

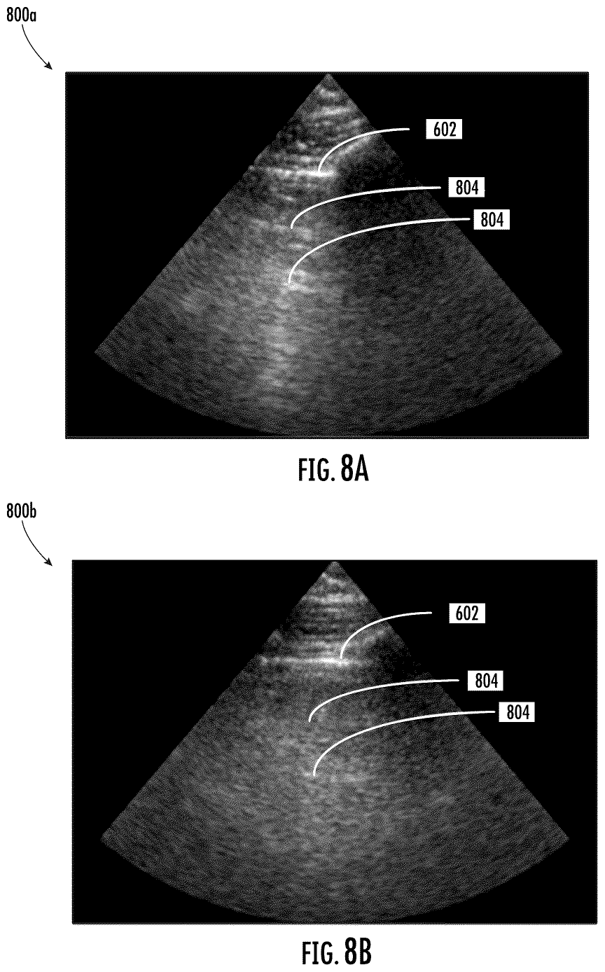

The probe 106 may include a transducer configured to transmit and receive an ultrasound signal. In some embodiments, as shown in FIG. 1, the probe 106 includes signal elements 108. The signal elements 108 may be arranged in a transducer array, and in some embodiments may be arranged in a 2D array (e.g., as illustrated by the matrix configuration 300 shown in FIG. 3). As described herein, the 2D array of the signal elements 108 may allow for 3D ultrasound imaging (e.g., such that the transducer is configured to transmit and receive a 3D ultrasound signal). The transmit beamformer 102 and the transmitter 104 drive the signal elements 108 to emit pulsed ultrasonic signals into a body of a subject (e.g., a patient). For example, during a lung ultrasound, a sonographer or other clinician may navigate the probe 106 over a patient's chest so that the signal elements 108 in the probe 106 emit the pulsed ultrasonic signals into the patient's thoracic cavity. The pulsed ultrasonic signals are then back-scattered from anatomical structures in the body, such as blood cells or muscular tissues, to produce echoes that return to the signal elements 108. During a lung ultrasound, however, there is a mismatch in the acoustic impedance between the lung (e.g., due to the lung being full of air) and the material of the transducer, meaning the ultrasonic signals are unable to back-scatter from the air-filled lung. Rather, the ultrasonic signals back-scatter from the pleura (e.g., causing generation of the pleura 602 in an ultrasound image of the lung, as described below with reference to FIGS. 6 and 8).

Additionally, in ultrasound imaging systems with electronically steered beams, the transmit beamformer 102 may be adjusted to shift an angle of the ultrasound beam, therefore shifting the ultrasound signal transmitted. For example, the transmit beamformer 102 may adjust timing and phase shifts of the ultrasound signals sent into the body of the subject to change the angle of the ultrasound signal. In various embodiments, the adjustment may occur without the operator having to move the transducer.

The receiver 110 receives the echoes from the probe 106 and converts the echoes into electrical signals. The electrical signals are then passed through the receive beamformer 112, which produces the ultrasound data from the electrical signals. As described above with reference to the transmit beamformer 102, the receive beamformer 112 may be either a hardware beamformer or a software beamformer. In embodiments where the receive beamformer 112 is a hardware beamformer, the receive beamformer 112 may include one or more of a GPU, a microprocessor, a CPU, a DSP, or any other type of processor capable of performing logical operations. The receive beamformer 112 may be configured to perform conventional beamforming techniques as well as techniques such as retrospective transmit beamforming (RTB). Alternatively, in embodiments where the receive beamformer 112 is a software beamformer, a processor (e.g., processor 116, as described below) may be configured to perform some or all of the functions associated with the receive beamformer 112.

Although the transmit beamformer 102, the transmitter 104, the receiver 110, and the receive beamformer 112 are shown in FIG. 1 as being components of the ultrasound imaging system 100 that are distinct from the probe 106, it should be appreciated that in some embodiments, the probe 106 may include electronic circuitry configured to perform the functions of each of the transmit beamformer 102, the transmitter 104, the receiver 110, and/or the receive beamformer 112. That is, all or part of the transmit beamformer 102, the transmitter 104, the receiver 110, and/or the receive beamformer 112 may be situated within the probe 106.

Referring still to FIG. 1, the ultrasound imaging system 100 is shown to include a processing circuit 114. As shown, the processing circuit 114 may include at least one processor 116, a memory 118, an image processing circuit 120, an artificial intelligence (AI) circuit 122, and a control circuit 124. In this way, the processing circuit 114 may be structured or configured to execute or implement the instructions, commands, and/or control processes described herein with respect to the processor 116, the memory 118, the image processing circuit 120, the AI circuit 122, and the control circuit 124. While shown as being separate from the probe 106 in FIG. 1, it will be appreciated that the processing circuit 114 can be part of the probe 106. For example, the processing circuit 114 can be disposed in a handheld housing of the probe 106 (e.g., in the case of the probe 106 being a wireless probe).

The processor 116 may include a CPU, a GPU, a microprocessor, a DSP, a general-purpose single- or multi-chip processor, a field-programmable gate array (FPGA), or any other type of processor capable of performing logical operations. A general-purpose processor may be a microprocessor, or, any conventional processor, or state machine. A processor also may be implemented as a combination of computing devices, such as a combination of a DSP and a microprocessor, a plurality of microprocessors, one or more microprocessors in conjunction with a DSP core, or any other such configuration. In some embodiments, the processor 116 may be shared by multiple circuits (e.g., the circuits of the processor 116 may include or otherwise share the same processor which, in some example embodiments, may execute instructions stored, or otherwise accessed, via different areas of the memory 118). Alternatively or additionally, the processor 116 may be structured to perform or otherwise execute certain operations independent of one or more co-processors. In some embodiments, two or more processors may be coupled via a bus to enable independent, parallel, pipelined, or multi-threaded instruction execution. All such variations are intended to fall within the scope of the present disclosure.

The processor 116 may be configured to control the transmit beamformer 102, the transmitter 104, the receiver 110, and the receive beamformer 112. The processor 116 may also be in electronic communication with the probe 106. For purposes of this disclosure, the term “electronic communication” may be defined to include both wired and wireless communications.

In some embodiments, the processor 116 may be configured to control the probe 106 during data acquisition. That is, the processor 116 may control the data acquisition by controlling which of the signal elements 108 are active and by controlling a shape of the beam emitted from the probe 106. For example, using the matrix configuration 300 of the signal elements 108 shown in FIG. 3, the processor 116 may be configured to control a transmit direction of the probe 106 such that data is acquired from a sagittal plane (e.g., azimuthal plane 210) and/or a transverse plane (e.g., elevation plane 215) of the probe 106. According to other embodiments, the transmit direction of the probe 106 may be controlled by another component of the ultrasound imaging system 100 (e.g., the control circuit 124).

Alternatively or additionally, the processor 116 may include a complex demodulator configured to demodulate radio frequency (RF) data obtained by the probe 106 and generate raw data. According to other embodiments, the demodulation of the RF data may be performed by another component of the ultrasound imaging system 100. The processor 116 may perform the processing operations described herein according to a plurality of selectable ultrasound modalities.

Depending on a mode of operation of the ultrasound imaging system 100, the processor 116 may process ultrasound data obtained by the probe 106 according to the mode of operation to generate image data. For example, the mode of operation may include B-mode, color flow Doppler mode, M-mode, color M-mode, spectral Doppler, elastography, TVI, strain, strain rate, and the like. Various of these modes of operation may be configured to, for instance, convert ultrasound data from beam space coordinates (e.g., received from the receive beamformer 112) to display space coordinates (e.g., such that the ultrasound data may be displayed as image data). In some embodiments, the mode of operation may allow for video processing by the processor 116 such that a series of images (e.g., processed ultrasound data) may be displayed in real-time while a scanning session/procedure is being performed on a patient. An operator of the ultrasound imaging system 100 (e.g., a sonographer) may switch between various modes to obtain a variety of ultrasound data and to perform a complete scan of an anatomical region of interest. For example, the operator may switch between modes using user interface 130 (e.g., using physical controls, interface inputs representing physical controls, etc.).

The processor 116 performs the processing operations in real-time as the echo signals are received by the receiver 110 from the probe 106. For the purposes of this disclosure, the term “real-time” is defined to include a procedure that is performed without any intentional delay. As an illustrative, non-limiting example, in certain instances, the ultrasound imaging system 100 may obtain images at a real-time volume-rate of 7-20 volumes/sec. It should be appreciated, however, that the real-time volume-rate may be dependent on the length of time that it takes to obtain each volume of data for display. Thus, the ultrasound imaging system 100 may be configured to obtain 2D data of an anatomical region at a faster rate than 3D data of the same anatomical region because it takes longer to obtain a volume of 3D data than the same volume of 2D data. Similarly, when the ultrasound imaging system 100 obtains a relatively large volume of data, the real-time volume-rate may be slower than for a smaller volume of data. For example, during an abdominal scan, the real-time volume-rate may be slower if the patient is an adult versus if the patient is an infant because the volume of data is larger for the adult than for the infant (e.g., due to the abdomen of an adult being larger than the abdomen of an infant). Therefore, certain implementations of the ultrasound imaging system 100 may have real-time volume-rates that are faster than 20 volumes/sec, while other implementations of the ultrasound imaging system 100 may have real-time volume-rates that are slower than 7 volumes/sec.

In some embodiments, the ultrasound imaging system 100 may include multiple processors configured to perform the processing operations/functionality described with reference to processor 116. For example, in such embodiments, a first processor of the multiple processors may be configured to demodulate and decimate the RF signal while a second processor of the multiple processors may be configured to further process the RF data prior to displaying an image representative of the data. It should be appreciated that other embodiments may use a different arrangement of processors.

The processor 116 may also be in electronic communication with the display device 132 such that the processor 116 may process ultrasound data obtained by the probe 106 and generate images to display on the display device 132 (e.g., first ultrasound image 600a, second ultrasound image 600b, first ultrasound image 800a, and second ultrasound image 800b as described below with reference to FIGS. 6A, 6B, 8A, and 8B).

As shown in FIG. 1, the processing circuit 114 also includes the memory 118. The memory 118 may be configured to, for example, store processed volumes of data obtained by the ultrasound imaging system 100 (e.g., ultrasound data collected by the probe 106). For example, the memory 118 may be a hospital picture archiving and communication system (PACS). The memory 118 (e.g., memory, memory unit, storage device, etc.) may include one or more devices (e.g., RAM, ROM, Flash memory, hard disk storage, etc.) for storing data and/or computer code for completing or facilitating the processes, layers, and modules described in the present application. The memory 118 may be or include tangible, non-transient volatile memory or non-volatile memory. The memory 118 may also include database components, object code components, script components, or any other type of information structure for supporting the activities and information structures described in the present application.

In various embodiments, the memory 118 may have varying capacity (e.g., storage space) across embodiments of the ultrasound imaging system 100. For example, the memory 118 may be configured to store at least 60 minutes' worth of ultrasound data. The ultrasound data may be stored in the memory 118 such that the ultrasound data may be retrieved according to an order/time of acquiring the data. That is, the ultrasound data may be stored with a timestamp indicating a time at which the ultrasound data was collected and may be retrieved starting with an oldest time at which the ultrasound data was collected.

The processing circuit 114 also includes the image processing circuit 120, the AI circuit 122, and the control circuit 124. Each of the image processing circuit 120, the AI circuit 122, and the control circuit 124 are configured to facilitate determination of the second transmit direction using the probe 106 during an ultrasound scan.

The image processing circuit 120 is configured to analyze ultrasound image data (e.g., obtained using the probe 106, stored in the memory 118, etc.) and identify anatomical structures, scanning planes, transmit directions, pathologies, and/or other features depicted by/contained within the image data. In some instances, the image processing circuit 120 may include multiple deep learning-based models configured to analyze the image data. Alternatively or additionally, the image processing circuit 120 may use multiple deep learning-based models included in the AI circuit 122 to analyze the image data, as described herein.

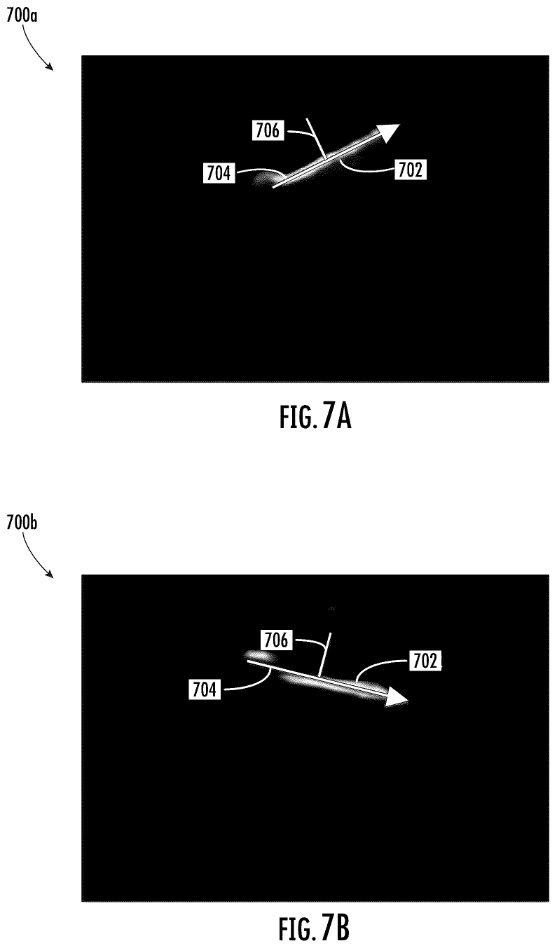

In some embodiments, the image processing circuit 120 may be configured to identify an anatomical structure, feature, region, etc. captured by the image data. For example, during a lung ultrasound, the image processing circuit 120 may be configured to identify the pleura, rib bones, shadows of the rib bones, and/or other pulmonary structures/features/regions depicted in the image data. The image processing circuit 120 may be configured to identify the anatomical structure using one or more algorithms (e.g., image processing algorithms such as edge detection, machine learning models, deep neural networks, etc.). In some embodiments, the image processing circuit 120 may be configured to apply one or more algorithms used by the AI circuit 122 and/or retrieved from the external database 128. For example, as described below with reference to FIGS. 7A, 7B, and 9-10B, the image processing circuit 120 may use one or more algorithms to assess the pleura 602 (e.g., using a segmentation model to segment the pleura 602, using linear regression to estimate pleural lines) captured by the image data. In this way, the image processing circuit 120 may be configured to estimate a direction of the pleura 602, and the transmit direction of the probe 106 may be adjusted based on the estimated direction with respect to the ultrasound beam such that the pleura can be scanned at a desired angle (e.g., at an angle where the pleura is perpendicular to the ultrasound beam), as described herein.

In some embodiments, the image processing circuit 120 may identify anatomical features such as bones, blood vessels, organs, etc., based on a shape, relative proximity, apparent depth, orientation, etc. of said features in the image data. Then, based on the identified anatomical features, the image processing circuit 120 may be configured to determine the anatomical structure depicted in the image data. For example, because a lung is not able to be imaged directly (e.g., due to the lung being full of air and the mismatch in the acoustic impedance between the air and the transducer of the probe 106), the image processing circuit 120 may determine that the lung is being imaged based on a movement and/or orientation of the lung relative to surrounding structures, such as the pleura, via a deep learning classification model trained to recognize the movement and/or orientation of the lung, and other anatomical structures. In some embodiments, the image processing circuit 120 may detect movement of structures in the image data by comparing the location, shape, size, etc., of identified structures across a set of images (e.g., a cine loop).

According to some embodiments, the image processing circuit 120 may be configured to identify a view or a scanning plane from which the ultrasound data is obtained. For example, the image processing circuit 120 may be configured to identify whether the image data depicts a sagittal view (e.g., along azimuthal plane 210) and/or a transverse view (e.g., along elevation plane 215.). Additionally, the image processing circuit 120 may be configured to identify a transmit direction from which the ultrasound data is obtained. For example, the image processing circuit 120 may be configured to use position data of the probe 106 to determine the transmit direction of the probe 106 with respect to an identified anatomical feature.

The image processing circuit 120 may also be configured to determine the presence of a pathology (e.g., an injury, disease, abnormality, etc.) in the image data. In some embodiments, the image processing circuit 120 may use a deep learning classification model trained to recognize various pathologies in the anatomical structure represented by the image data to specify the pathology that is present. Continuing with the example of the lung ultrasound, the image processing circuit 120 may use a deep learning classification model trained to recognize pulmonary pathologies to determine whether a pathology is present in the image data from the lung ultrasound. For instance, the image processing circuit 120 may identify pleural effusion (e.g., a buildup of fluid) in the pleura as a pulmonary pathology present in the image data. In some embodiments, the AI circuit 122 may be configured to perform any of the functions of the image processing circuit 120 described herein using multiple deep learning-based models configured to analyze the image data.

Based on the analysis of the image data, processing circuit 114 (e.g., the AI circuit 122) may be configured to determine an adjustment to the probe 106. In some embodiments, the adjustment to the probe 106 may include a change in the angle of the probe 106. For example, during a lung ultrasound, the image processing circuit 120 may determine an estimated angle of the pleura with respect to the ultrasound beam (e.g., using a plurality of images obtained from a sagittal plane and a transverse plane), and the processing circuit 114 may determine, as described below with reference to step 520 of method 500, a transmit direction to which the angle of the probe 106 is adjusted to (e.g., by adjusting the transmit beamformer 102). As another example, the control circuit 124 may determine a transmit direction to which the probe 106 should be adjusted to for better image quality and the processing circuit 114 may display, as described below with reference to step 530 of method 500, the transmit direction such that an operator of the ultrasound imaging system 100 may adjust the probe 106. In this way, the quality of the anatomical feature (e.g., the pleura 602) may be improved due to bi-plane images (e.g., from the sagittal view and from the transverse view) obtained by the probe 106 being analyzed to determine the current angle of the anatomical feature with respect to the probe 106 and adjust the current angle towards a desired angle (e.g., such that image quality is improved, as is shown in FIGS. 8A and 8B).

In some embodiments, the processing circuit 114 (e.g., the AI circuit 122) may be configured to automatically generate a control signal prompting an adjustment of the probe 106 based on the analysis of the image data performed by the image processing circuit 120. The control signal may be received by the control circuit 124, which may be configured to automatically align the transmit direction of the probe 106 such that the probe 106 is configured to acquire image data according to the transmit direction prescribed by the control signal. For example, the control signal may prompt the control circuit 124 to adjust the transmit beamformer 102, thereby adjusting the ultrasound beam, to shift the transmit direction of the probe 106.

The ultrasound imaging system 100 may also include an external database 128 and a user interface 130. The external database 128 refers to a database from which the processing circuit 114 (e.g., the image processing circuit 120, the AI circuit 122) retrieves information used in bi-plane imaging during a lung ultrasound. For example, the external database 128 may be a medical information database. The medical information database may store clinical guidelines, standard practices, medical literature, medical textbooks, published research, previous case studies, and so on. Depending on an implementation of the ultrasound imaging system 100 and/or a procedure performed thereby, the processing circuit 114 may retrieve clinical guidelines, standard practices, medical literature, medical textbooks, published research, and previous case studies related to the implementation and/or procedure. For example, if the ultrasound imaging system 100 is being used in a hospital setting to conduct a lung ultrasound, the processing circuit 114 may retrieve clinical guidelines and standard practices related to the hospital setting and the lung ultrasound. Continuing with this example, the processing circuit 114 may also retrieve information from the medical literature, medical textbooks, published research, and previous case studies related to pulmonary anatomy and the lung ultrasound. In some instances, as described with reference to FIGS. 7A, 7B, and 9-10B, the information retrieved from the external database 128 may include one or more algorithms used to segment the anatomical feature and determine a vector that represents the anatomical feature (e.g., segmenting the pleura 602 and determining a 3D vector using an algorithm).

The user interface 130 may be used by a sonographer or other clinician to control operation of the ultrasound imaging system 100. For example, the sonographer may use the user interface 130 to control the input of patient data, to change a scanning or display parameter, and/or to select various other modes, operations, parameters, etc. of the ultrasound imaging system 100. In some embodiments, the user interface 130 may include an off-the-shelf consumer electronic device such as a smartphone, a tablet, a laptop, and so on. For the purposes of this disclosure, the term “off-the-shelf consumer electronic device” is defined to be an electronic device that was designed and developed for general consumer use and one that was not specifically designed for use in a medical environment. Alternatively, in other embodiments, the user interface 130 may be an electronic device that was designed and developed for use in a medical environment.

According to some embodiments, the user interface 130 may be physically separate from the rest of the ultrasound imaging system 100 (e.g., the transmit beamformer 102, the transmitter 104, the probe 106, the receiver 110, the receive beamformer 112, the processing circuit 114, and/or the external database 128). The user interface 130 may communicate with the processor 116 through a wireless protocol, such as Wi-Fi, Bluetooth, wireless local area network (WLAN), near-field communication, and so on. According to some embodiments, the user interface 130 may communicate with the processor 116 through an application programming interface (API).

In some embodiments, the user interface 130 may include physical controls such as one or more of buttons, sliders, a rotary knob, a mouse, a keyboard, a trackball, hard keys linked to specific actions, soft keys that may be configured to control different functions, and so on. As shown in FIG. 1, the user interface 130 may also include a display device 132. In some embodiments, the display device 132 may be configured to display a graphical user interface (GUI) based on an instruction from the memory 118. The GUI may include user interface icons representing commands and instructions relating to the operation of the ultrasound imaging system 100. The user interface icons of the GUI may be configured such that a user (e.g., the sonographer, clinician, etc.) may select a specific user interface icon to initiate a specific function controlled by the GUI. For example, various user interface icons may be used to represent windows, menus, buttons, cursors, scroll bars, and so on. That is, the physical controls of the user interface 130 may be included as individual hardware elements, as user interface icons displayed on the display device 132, or as a combination of hardware elements and user interface icons.

In some embodiments, the display device 132 may include a touch-sensitive display device or a touch screen. According to such embodiments, the touch screen may be configured to interact with the GUI displayed by the display device 132 such that a user (e.g., the sonographer) can interact with the GUI via the touch screen. The touch screen may be a single-point touch screen that is configured to detect a single contact point at a time, or the touch screen may be a multi-point touch screen that is configured to detect multiple points of contact at a time. For embodiments where the touch screen is a multi-point touch screen, the touch screen may be configured to detect multi-point gestures involving contact from two or more of a user's fingers at a time. The touch screen may be a resistive touch screen, a capacitive touch screen, or any other type of touch screen that is configured to receive inputs from a stylus or one or more of a user's fingers. According to some embodiments, the touch screen may be an optical touch screen that uses technology such as infrared light or other frequencies of light to detect one or more points of contact initiated by a user. In some embodiments, the touch screen may be incorporated as part of the display device 132 or may be separate from the display device 132. The user interface 130 may also include a proximity sensor configured to detect objects and/or gestures that are within a predetermined distance (e.g., five feet, six inches, ten centimeters, etc.) of the proximity sensor. In various embodiments, the proximity sensor may be located on the display device 132 or as part of a touch screen that is separate from the display device 132.

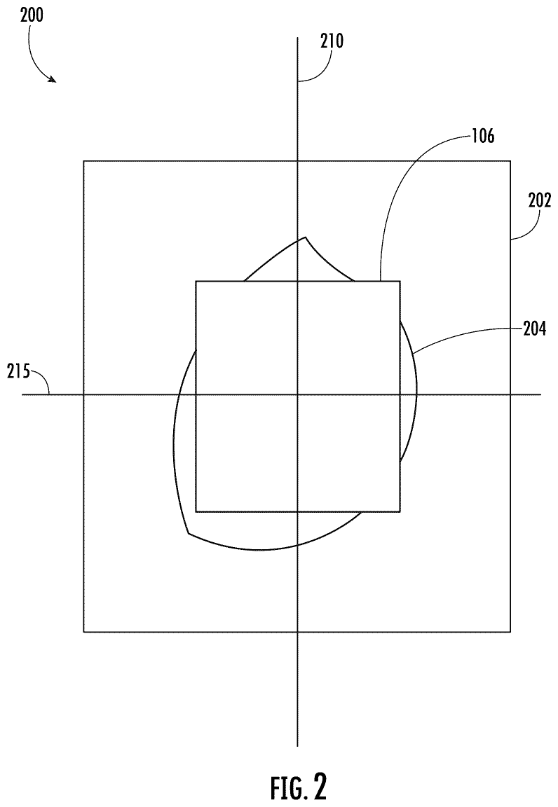

Referring to FIG. 2, an orientation 200 of the probe 106 is shown. The orientation 200 shown in FIG. 2 refers to a desired orientation of the probe 106 during a lung ultrasound, as described herein. More specifically, the orientation 200 refers to an orientation of the probe 106 relative to a field of view 202 which includes a pleura 204. As shown, the orientation 200 may be defined by two orthogonal planes including an azimuthal plane 210 and an elevation plane 215. The azimuthal plane 210 captures image data from a sagittal view of the probe 106, and the elevation plane 215 captures image data from a transverse view of the probe 106. According to the orientation 200, the azimuthal plane 210 and the elevation plane 215 are shown perpendicular to the pleura 204. During a lung ultrasound, it may be desired to orient the probe 106 such that the ultrasound beam in the azimuthal plane 210 and the elevation plane 215 are perpendicular to the pleura 204, resulting in an image with optimal quality.

When positioning the probe 106 on the patient, a position of the probe 106 is naturally perpendicular to the body of the patient. However, the pleura 204 may not be parallel to the body such that the pleura 204 is not perpendicular to the probe 106, as suggested by FIG. 2, but rather can be shifted at an angle (e.g., as represented in FIGS. 6A and 6B). When acoustic transmission is parallel to the probe 106, depending on the angle of the pleura 602, an image captured may have a degraded image quality, as described herein. Moreover, the positioning of the patient's pleura may be unique to an individual anatomy of the patient, meaning the position of a first patient's pleura may differ from a position of a second patient's pleura, and so on. This may be corrected by reorienting acoustic transmission of the probe 106, therefore adjusting the ultrasound signal transmitted to be as perpendicular to the pleura. This may also be corrected, by a user of the probe 106 reorienting the probe 106. Rather than relying on the expertise/skill of the sonographer to orient the probe 106 such that the pleura 602 is perpendicular to the ultrasound beam in the azimuthal plane 210 and the elevation plane 215, the transmit direction of the probe 106 may be automatically adjusted, as described herein, such that the probe 106 is configured to capture ultrasound images from a direction where the pleura is substantially perpendicular to the ultrasound beam.

Referring to FIG. 3, a matrix configuration 300 of the ultrasound probe 106 is shown. The matrix configuration 300 refers to a configuration of the signal elements 108 within the ultrasound probe 106. More specifically, the matrix configuration 300 may include a 2D array of the signal elements 108 such that the probe 106 is configured to capture image data from several planes (e.g., a sagittal plane, an elevation plane, a transverse plane, etc.). In other words, with the matrix configuration 300, the probe 106 may obtain ultrasound images from a plurality of scanning planes. More specifically, as described herein, the probe 106 may be configured to obtain ultrasound images of a patient's lung from a sagittal plane and/or a transverse plane at a number of angles between an anatomical feature and the probe 106 and/or the ultrasound between. According to an example configuration of the probe 106, the matrix configuration 300 may include a 2D array of 6,000 elements (e.g., signal elements 108).

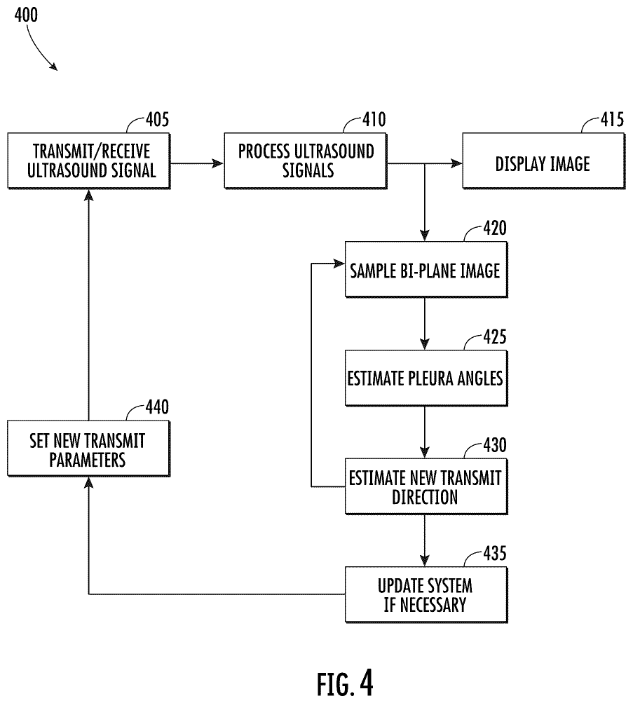

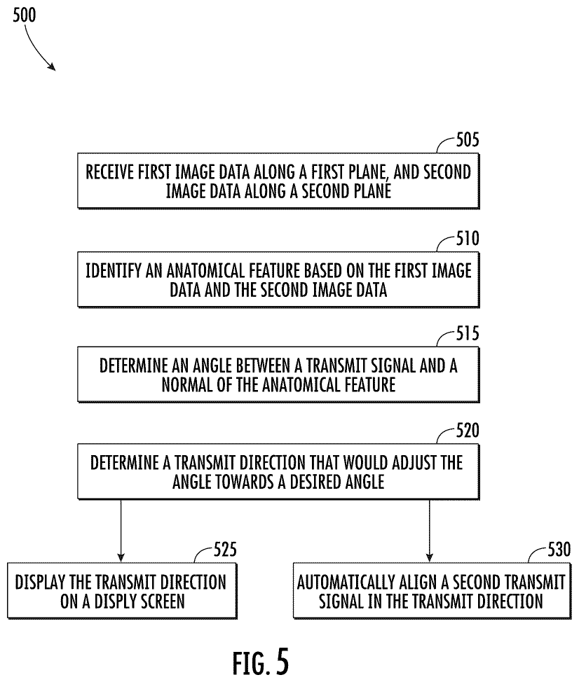

As described herein, with the matrix configuration 300, the orientation of the probe 106 may be adjusted with respect to the pleura (e.g., using method 400 and/or 500 as described below with reference to FIGS. 4 and 5). In other words, although at an initial orientation of the probe 106, the azimuthal plane 210 and the elevation plane 215 show that the probe 106 and/or the ultrasound beam are not perpendicular to pleura, the matrix configuration 300 allows the probe 106 to be adjusted to capture image data in which the probe 106 and/or the ultrasound beam is perpendicular to the pleura based on the anatomy of the patient being imaged during the lung ultrasound.

Referring to FIG. 4, a flow chart illustrating a method 400 for bi-plane ultrasound imaging using an ultrasound imaging system is shown. In at least one embodiment, the ultrasound imaging system referred to by method 400 is the ultrasound imaging system 100 described above with reference to FIG. 1, and method 400 may be implemented by the ultrasound imaging system 100. In some embodiments, method 400 may be implemented as executable instructions in a memory of the ultrasound imaging system 100, such as the memory 118 of FIG. 1.

As shown in FIG. 4, at step 405, method 400 includes transmitting and receiving an ultrasound signal. The ultrasound signal may be transmitted and received in a first plane and a second plane. An ultrasound beam refers to the transmitted ultrasound signal. In various embodiments, movement of the probe 106 can adjust the ultrasound beam. In other embodiments the ultrasound imaging system 100 may be configured to electronically adjust the ultrasound beam (e.g., by using the processing circuit 114). The first plane is orthogonal to the second plane. In some embodiments, as described herein, the first plane refers to a sagittal plane (e.g., azimuthal plane 210) and the second plane refers to a transverse plane (e.g., elevation plane 215). As disclosed herein, the transmit beamformer 102 drives the signal elements 108 to emit pulsed ultrasonic signals. The transmit beamformer 102 and/or the probe 106 can be adjusted to adjust the ultrasound beam.

Step 410 of method 400 includes processing ultrasound signals. Processing of the ultrasound signals allows for image construction. Processing ultrasound signals may include converting the received echoes from the probe 106 into electrical signals, passing the electrical signals through the receive beamformer 112 for ultrasound data, and performing beamforming techniques to generate an image. The processing of ultrasound signals occurs in both the first plane and second plane (e.g., FIGS. 6A and 6B depict constructed ultrasound images) to receive a first image data and a second image data. In various embodiments, the image processing circuit 120 applies processing techniques to the constructed image to further improve quality of the constructed images (e.g., contrast adjustment, brightness adjustment, noise reduction, etc.).

At step 415, a bi-plane image is displayed with the first image data (e.g., received at step 410) and the second image data (e.g., received at step 410). In some embodiments, the bi-plane image refers to a 3D ultrasound image depicting ultrasound data obtained from the sagittal view (e.g., along the azimuthal plane 210) and from the transverse view (e.g., along the elevation plane 215). In some embodiments, the bi-plane image may be displayed via the display device 132. Although the systems and methods described herein refer to exemplary embodiments in which the two planes refer to a sagittal plane and a transverse plane, it should be appreciated that this disclosure is not limited to such operation and that the various systems and methods are configured to apply to any variety of planes and/or orientations.

At step 420, the first image data and the second image data is sampled to identify an anatomical feature in both image data. In some embodiments, step 420 may be performed by the processing circuit 114. For example, as described herein, the first image data and the second image data may be obtained during a lung ultrasound, and therefore the anatomical feature may include a pleura of the patient being imaged. It should be appreciated, however, that although the ultrasound imaging system 100 cannot capture an image of the lung cavity due to the lung cavity being filled with air, an image of the lung cavity may be derived using surrounding anatomical structures/features (e.g., ribs, the pleura 602, etc.) depicted in the first image data and the second image data. In some embodiments, step 420 may also include detecting a pathology in the first image data and the second image data (e.g., using the image processing circuit 120, as described above).

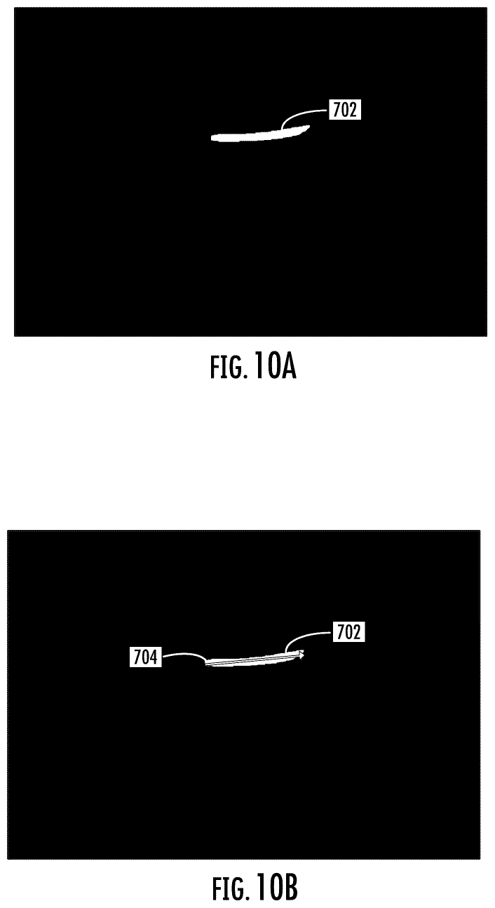

Identifying the anatomical feature may include segmenting the anatomical feature. Segmentation of the anatomical feature can produce a binary mask of the anatomical feature (e.g., FIG. 10A depicts a binary mask of the pleura). Although various methods can be employed to segment the anatomical feature, one way to do so is by training a segmentation model (e.g., U-Net, Mask R-CNN, DeepLab, etc.). The trained model can analyze an image data (e.g., the first image data, the second image data) and output a probability for each data point (e.g., pixel) of the image data being the anatomical feature or a background, i.e., not the anatomical feature. Setting a threshold for the probabilities to determined which pixels classify as the anatomical feature then creates the binary mask of the anatomical feature for identification.

At step 425, method 400 includes determining an angle between the ultrasound beam and a normal of the anatomical feature. This is done by first characterizing the identified anatomical feature in the first image data and in the second image data as a first vector and a second vector, respectively, to determine the normal of the anatomical feature. A number of methods can be employed to fit the identified anatomical feature into a best-fit line. For example, orthogonal distance regression (ODR) can be used to calculate the best-fit line and determine the first vector and the second vector. ODR minimizes a sum of the squared orthogonal distances from each data point (e.g., the white pixels in the mask illustrated in FIG. 10A) of the anatomical feature to form a vector. For example, in some embodiments, ODR minimizes the sum of the squared orthogonal distances using the following equation:

$${\sum }_{i = 1}^{n} {( \frac{\vert {mx}_{i} - {y}_{i} + b \vert}{\sqrt{{m}^{2} + 1}} )}^{2} , \qquad ( Eq . 1 )$$

where n is a total number of data points, m is the slope of the anatomical feature, b is the intercept with the Y axis, and for the i-th data point, xᵢ is the corresponding x-position and yᵢ is the corresponding y-position. In this way, the parameters m and b together can define the best-fit line, and ORD can be used to assess those two values based on a set of points (e.g., xᵢ, yᵢ).

Although the first vector and the second vector are shown in a 2D space, the first vector and the second vector may be expanded to a 3D vector (e.g., by adding a zero in either an X or Y dimension, dependent on the plane). The normal of the anatomical feature can be determined by determining a vector that is perpendicular to both the first vector and the second vector. Cross product calculation of the first vector and the second vector can be used to determine the normal of the anatomical feature.

With the normal of the anatomical feature determined, the angle between the ultrasound beam and the normal of the anatomical feature may be determined. The angle may be determined using trigonometric functions (e.g., arccosine). For example, in some embodiments, the angle may be determined using the following equation:

$$α = {cos}^{- 1} ( {v}_{0} \cdot \frac{{v}_{1} \times {v}_{2}}{‖ {v}_{1} \times {v}_{2} ‖} ) , \qquad ( Eq . 2 )$$

where v₀ is a vector defining the ultrasound beam, v₁ is the first vector and v₂ is the second vector. In various embodiments, the vector defining the ultrasound beam is directed perpendicular to a surface being ultrasound (e.g., the vector is (0,0,−1) in a Cartesian coordinate system) and:

$$α = {cos}^{- 1} ( ( 0 , 0 , - 1 ) \cdot \frac{{v}_{1} \times {v}_{2}}{‖ {v}_{1} \times {v}_{2} ‖} ) = {cos}^{- 1} ( \frac{1}{\sqrt{{m}_{1}^{2} + {m}_{2}^{2} + 1}} ) , \qquad ( Eq . 3 )$$

where m₁ is the slope of the first vector in the first plane and m₂ is the slope of the second vector in the second plane.

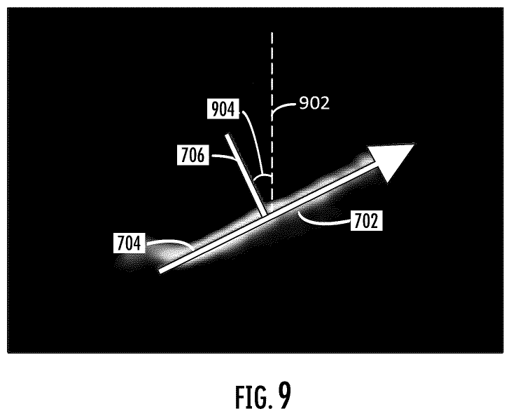

Position of the ultrasound beam and/or the probe 106 for the angle calculation may be determined using position data from the probe 106. In various embodiments, the probe 106 and the ultrasound beam are positioned towards the body such that the direction is (0,0,−1) in a Cartesian coordinate system. In some embodiments, an angle between the ultrasound beam and a normal of each vector (e.g., the first vector, the second vector) is determined (e.g., FIG. 9 as further described below). The determination of the angles in the first plane and the second plane can aid in determining how the probe must be adjusted.

In various embodiments, steps 405-425 are performed using a 3D scan. At step 405, the probe 106 can be configured for 3D scanning and transmit and receive ultrasound signals along multiple planes and angles. At step 410, as described herein, the ultrasound signals are processed for image construction to form a 3D image. The 3D scan can capture a volumetric dataset that includes information in three dimensions. As described in respect to step 420, 3D segmentation can be used to identify the anatomical feature. For example, a 3D network (e.g., 3D convolutional neural networks, projective convolutional networks, voxel-based networks, etc.) can be used to estimate data points (e.g., voxels) of the anatomical feature. Step 425 can include characterizing the identified anatomical feature in the 3D scan. A number of methods can be employed to fit the identified anatomical feature into a best-fit plane. For example, Principal Component Analysis (PCA) or least square fitting can used. Fitting the identified anatomical feature may provide a simpler representation of the identified anatomical feature for forming the first vector and the second vector. From the best-fit plane, the first vector and the second vector can be extracted. This may be done by extracting the first plane and the second plane from the 3D scan. The best-fit plane of the identified anatomical feature forms the first vector along the first plane at a corresponding position of the best-fit plane and forms the second vector along the second plane at a corresponding position of the best-fit plane.

At step 430, a transmit direction of the probe and/or the ultrasound beam that would adjust the angle between the ultrasound beam and the normal of the anatomical feature to a desired angle is determined. For example, for a pleura, an optimal orientation of the ultrasound beam is perpendicular to the pleura. Such, the desired angle between the ultrasound beam and the normal of the pleura would be 0°. In various embodiments, the processing circuit 114 performs step 430. In various embodiments, the angle between the ultrasound beam and the normal of the anatomical feature in the first plane and in the second plane is used to determine how the probe and/or the ultrasound beam must be adjusted to reach the desired angle.

Method 400 continues at step 435, by updating ultrasound imaging system 100 if necessary. For example, the processing circuit 114 may determine that the angle between the ultrasound beam and the normal of a pleura is substantially at 0°, which is a desired angle. Thus, no update will be needed to the ultrasound imaging system 100. In some embodiments, the processing circuit 114 may determine that the angle is at the desired angle within a margin of error (e.g., the desired angle of 0°±5°). The processing circuit 114 may determine that the angle between the ultrasound beam and the normal of a pleura is not substantially at the desired angle and needs to be updated. For example, FIGS. 6A and 6B show a bi-plane image in which the angle is not substantially at the desired angle of 0°, but at 29.0°. Based on the determination, the transmit direction determined in step 435 is used to update the ultrasound imaging system 100.

At step 440, new transmit parameters are set. The transmit direction can be displayed on the user interface 130 of the ultrasound imaging system 100, such that an operator can use the displayed transmit direction to adjust the probe 106. As disclosed herein, the user interface 130 may also provide an audio output prompting the operator to adjust the probe 106. In some embodiments, the instruction could prompt the operator to tilt the probe 106 by X degrees along an axis in a first direction or by Y degrees along the axis in a second direction opposite of the first direction such that the tilting of the probe 106 results in alignment of a second ultrasound signal to the transmit direction determined at step 435. For example, aligning a second ultrasound signal to the transmit direction may require the user to tilt the probe by 10°. Other movements of the probe 106, including rotating, sliding, rocking, etc., may be provided to the operator to align the second ultrasound signal to the transmit direction. The probe 106 may be automatically aligned (e.g., if the probe 106 is capable of making such an adjustment) in the transmit direction. For example, automatically aligning a second ultrasound signal may include controlling which of the signal elements 108 included in the matrix configuration 300 are active such that the active signal elements 108 from the matrix configuration 300 are configured to collect ultrasound data along the transmit direction determined at step 435. In some embodiments, the transmit beamformer 102 is adjusted to adjust the second ultrasound signal to the transmit direction.

Method 400 continues by repeating step 405 using the new transmit direction to transmit and receive a second ultrasound signal in the first plane and the second plane. The second ultrasound signals are then processed in step 410 to allow for image construction. At step 415, a second bi-plane image is displayed with the image data received from the second ultrasound signal to display the anatomical feature at the desired angle (e.g., FIGS. 8A and 8B depict constructed ultrasound images in which the anatomical feature is at the desired angle). In such a way, the ultrasound imaging system 100 can provide real-time guidance for generating bi-plane images during an ultrasound scan. In various embodiments, the ultrasound imaging system 100 can provide continuous guidance by repeating steps 420-440 the new transmit direction to continuously optimize a displayed image of the anatomical feature.

Referring to FIG. 5, a flow chart is shown illustrating a method 500 for angle estimation and ultrasound probe adjustment during the method 400. In at least one embodiment, the ultrasound probe referred to by method 500 is the ultrasound probe 106 described above with reference to FIG. 1, and method 500 may be implemented by the ultrasound imaging system 100. In some embodiments, method 500 may be implemented as executable instructions in a memory of the ultrasound imaging system 100, such as the memory 118 of FIG. 1.

As shown in FIG. 5, method 500 includes receiving a first image data along a first plane and a second image data along a second plane at step 505. The first plane is orthogonal to the second plane. In some embodiments, as described herein, the first plane refers to a sagittal plane (e.g., azimuthal plane 210) and the second plane refers to a transverse plane (e.g., elevation plane 215). In some embodiments, step 505 of method 500 may occur after completion or during step 410 of method 400. For example, FIGS. 6A and 6B depict a bi-plane image construction of a received first image data and second image data along the first plane and the second plane accordingly.

As shown in FIG. 5, method 500 continues with identifying the anatomical feature based on the first image data and the second image data at step 510. In some embodiments, step 510 occurs during step 420 of method 400 (e.g., sampling bi-plane image constructed with the first image data and the second image data). The processing circuit 114 (e.g., the image processing circuit 120, the AI circuit 122, etc.) may be configured to identify the anatomical feature using segmentation models (e.g., U-Net, Mask R-CNN, DeepLab). The identified anatomical feature may be displayed as a binary mask (e.g., FIG. 10A depicts a binary mask of a pleura). Additionally, the identified anatomical feature may be represented as a 2D or a 3D vector using a best-fit line as disclosed herein (e.g., FIGS. 7A, 7B, 9, and 10B depict binary masks of a pleura with a best-fit line). For example, ODR may be used to determine the best-fit line by minimizing Eq. 1 as disclosed in reference to step 425.

After identification of the anatomical feature, method 500 continues with determining an angle between the ultrasound beam from the ultrasound imaging system 100 and a normal of the anatomical feature at step 515. The normal of the anatomical feature may be determined by using the first image data and the second image data (e.g., using a 3D vector of the anatomical feature in the first image data and the second image data to determine a vector normal to both using a cross product calculation). With the calculation of the normal of the anatomical feature, trigonometric functions (e.g., arccosine) may be used to determine the angle between the ultrasound beam and the normal of the anatomical feature. For example, Eq. 2 as disclosed in reference to step 425 may be used to determine the angle. The angle can be used to determine an image quality of any ultrasound images received with the probe positioned at the angle with respect to the pleura. For example, with a desired angle between a normal of the pleura and the ultrasound beam being 0° such that the ultrasound beam is perpendicular to the pleura, a determined angle that is substantially not 0° would represent an image of low quality.

At step 520, a transmit direction that would adjust the angle determined in step 520 to a desired angle is determined. In some embodiments, the transmit direction is determined at step 430 of method 400. In various embodiments, the angle between the ultrasound beam and the normal of the anatomical feature in both the first image data and in the second image data can be used to determine the transmit direction along the first plane and the second plane to reach the desired angle (e.g., determination of an azimuth angle and an elevation angle). The angle in the first image data and the second image data may be determined using trigonometric functions (e.g., arctangent). For example, in some embodiments, the angle may be determined by the following equation:

$$α = {tan}^{- 1} ( m ) , \qquad ( Eq . 4 )$$

where for the first image data determination, m is the slope of the anatomical feature in the first plane, and for the second image data determination, m is the slope of the anatomical feature in the second plane.

Using the transmit direction determined in step 520, method 500 may continue to step 525 and/or step 530. At step 525, the transmit direction is displayed on a display device 132. The displayed transmit direction can be used by an operator to adjust the probe 106. For example, the operator could be prompted by the display device 132 to tilt the probe 106 by X degrees along an axis in a first direction or by Y degrees along the axis in a second direction opposite of the first direction such that the tilting of the probe 106 results in alignment of a second ultrasound signal to the transmit direction determined at step 435. For example, aligning a second ultrasound signal to the transmit direction may require the user to tilt the probe by 10°. Other movements of the probe 106, including rotating, sliding, rocking, etc., may be provided to the operator to align the second ultrasound signal to the transmit direction. In some embodiments, the user interface 130 could prompt the operator to adjust the probe 106 by providing an audio output.

At step 530, a second ultrasound signal is automatically aligned in the transmit direction. Automatically aligning a second ultrasound signal may include controlling which of the signal elements 108 included in the matrix configuration 300 are active such that the active signal elements 108 from the matrix configuration 300 are configured to collect ultrasound data along the transmit direction. Additionally, the transmit beamformer 102 may be automatically adjusted to adjust the ultrasound beam, therefore adjusting the ultrasound signal, along the transmit direction.

Referring to FIGS. 6A-6B, a bi-plane image of a first ultrasound image 600a acquired in a first plane and a second ultrasound image 600b acquired in a second plane is shown. The first plane is orthogonal to the second plane (e.g., an azimuth plane and an elevation plane). The first ultrasound image 600a and the second ultrasound image 600b may be acquired using techniques described in FIGS. 4 and 5 (e.g., steps 405-415 and 505). The first ultrasound image 600a and the second ultrasound image 600b show the pleura 602. In the images, A-lines of a lung of the images cannot be visualized and the image quality is not of optimal quality which may suggest that an orientation of the probe 106 for image collection of the bi-plane image was not optimal (e.g., for the bi-plane image of FIGS. 6A-6B, an angle between a normal of the pleura 602 and the ultrasound beam was determined to be 29°). A first image data and a second image data from the first ultrasound image 600a and the second ultrasound image 600b, respectively can be used to determine how the probe 106 and/or the ultrasound beam must be adjusted to obtain a second bi-plane image at an optimal quality.

Referring to FIGS. 7A-7B, images of the first ultrasound image 600a and the second ultrasound image 600b after image processing (e.g., steps 420 and/or 510) are shown. The pleura 602 in the first ultrasound image 600a and the second ultrasound image 600b are segmented, depicted by a segmented pleura 702. Segmentation of the pleura 602 can be performed using segmentation models disclosed herein. Data points (e.g., pixels) of the segmented pleura 702 are fit onto a line to create a vector 704. The vector 704 is used to determine a normal of the pleura 602 (e.g., by calculating a cross product of the vector 704 in both images to determine a normal vector, i.e., the normal of the pleura 602, perpendicular to both vectors 704). As disclosed herein (e.g., at steps 430 and 515-520), the normal of the pleura 602 may be used to determine an angle between the normal of the pleura and the ultrasound beam, which allows for the determination of a transmit direction to adjust a second ultrasound signal to a desired angle to receive a third image data set. A normal 706 of the segmented pleura 702 in each of the images may be used to determine the transmit direction (e.g., as shown in FIG. 9) and determine how the probe 106 and/or the ultrasound beam should be adjusted to obtain the desired angle between the normal of the anatomical feature and the ultrasound beam.

Referring to FIGS. 8A-8B, a second bi-plane image of a first ultrasound image 800a acquired in the first plane and a second ultrasound image 800b acquired in the second plane is shown. The second bi-plane image is constructed using the second ultrasound signal determined after image processing as disclosed herein (e.g., with reference to the images of FIGS. 7A-7B). The second ultrasound signal is acquired along the transmit direction such that the angle between the normal of the pleura 602 and the ultrasound beam and/or the probe 106 is closer to a desired angle, which for the pleura 602 is 0° (e.g., for the bi-plane image of FIGS. 8A-8B, an angle between a normal of the pleura 602 and the probe 106 was determined to be 4.2°). In comparison to FIGS. 6A-6B, FIGS. 8A-8B show an improvement in image quality as the pleura 602 appears brighter and A-lines 804 can be visualized in the first ultrasound image 800a and the second ultrasound image 800b.

Referring to FIG. 9, an image of the first ultrasound image 600a after image processing (e.g., steps 420 and/or 510) is shown. The normal 706 of the segmented pleura 702 in the image may be used to determine the transmit direction. For example, an angle 904 between the normal 706 of the pleura segmented pleura 702 and an ultrasound beam 902 can be calculated and compared with a desired angle to determine how the ultrasound beam 902 must shift to align with the transmit direction needed to have the desired angle.

Referring to FIG. 10A-10B, segmentation and vector determination of a pleura is shown according to an exemplary embodiment. Specifically, FIG. 10A shows a binary mask (the segmented pleura 702) of the pleura created using segmentation processes as disclosed in step 420 of method 400. The binary mask is then used to create a vector 704, shown in FIG. 10B. Determination of the vector 704 may occur using the processes disclosed in step 425 of method 400.

The embodiments described herein have been described with reference to drawings. The drawings illustrate certain details of specific embodiments that provide the systems, methods and programs described herein. However, describing the embodiments with drawings should not be construed as imposing on the disclosure any limitations that may be present in the drawings.

It should be understood that no claim element herein is to be construed under the provisions of 35 U.S.C. § 112(f), unless the element is expressly recited using the phrase “means for.”

As utilized herein, terms of degree such as “approximately,” “about,” “substantially,” and similar terms are intended to have a broad meaning in harmony with the common and accepted usage by those of ordinary skill in the art to which the subject matter of this disclosure pertains. It should be understood by those of skill in the art who review this disclosure that these terms are intended to allow a description of certain features described and claimed without restricting the scope of these features to any precise numerical ranges provided. Accordingly, these terms should be interpreted as indicating that insubstantial or inconsequential modifications or alterations of the subject matter described and claimed are considered to be within the scope of the disclosure as recited in the appended claims.

It should be noted that terms such as “exemplary,” “example,” and similar terms, as used herein to describe various embodiments, are intended to indicate that such embodiments are possible examples, representations, or illustrations of possible embodiments, and such terms are not intended to connote that such embodiments are necessarily extraordinary or superlative examples.

The term “coupled” and variations thereof, as used herein, means the joining of two members directly or indirectly to one another. Such joining may be stationary (e.g., permanent or fixed) or moveable (e.g., removable or releasable). Such joining may be achieved with the two members coupled directly to each other, with the two members coupled to each other using a separate intervening member and any additional intermediate members coupled with one another, or with the two members coupled to each other using an intervening member that is integrally formed as a single unitary body with one of the two members. If “coupled” or variations thereof are modified by an additional term (e.g., directly coupled), the generic definition of “coupled” provided above is modified by the plain language meaning of the additional term (e.g., “directly coupled” means the joining of two members without any separate intervening member), resulting in a narrower definition than the generic definition of “coupled” provided above. Such coupling may be mechanical, electrical, or fluidic.

The term “or,” as used herein, is used in its inclusive sense (and not in its exclusive sense) so that when used to connect a list of elements, the term “or” means one, some, or all of the elements in the list. Conjunctive language such as the phrase “at least one of X, Y, and Z,” unless specifically stated otherwise, is understood to convey that an element may be either X, Y, Z; X and Y; X and Z; Y and Z; or X, Y, and Z (i.e., any element on its own or any combination of X, Y, and Z). Thus, such conjunctive language is not generally intended to imply that certain embodiments require at least one of X, at least one of Y, and at least one of Z to each be present, unless otherwise indicated.

References herein to the positions of elements (e.g., “top,” “bottom,” “above,” “below”) are merely used to describe the orientation of various elements in the drawings. It should be noted that the orientation of various elements may differ according to other exemplary embodiments, and that such variations are intended to be encompassed by the present disclosure.

As used herein, terms such as “engine” or “circuit” may include hardware and machine-readable media storing instructions thereon for configuring the hardware to execute the functions described herein. The engine or circuit may be embodied as one or more circuitry components including, but not limited to, processing circuitry, network interfaces, peripheral devices, input devices, output devices, sensors, etc. In some embodiments, the engine or circuit may take the form of one or more analog circuits, electronic circuits (e.g., integrated circuits (IC), discrete circuits, system on a chip (SOCs) circuits, etc.), telecommunication circuits, hybrid circuits, and any other type of circuit. In this regard, the engine or circuit may include any type of component for accomplishing or facilitating achievement of the operations described herein. For example, an engine or circuit as described herein may include one or more transistors, logic gates (e.g., NAND, AND, NOR, OR, XOR, NOT, XNOR, etc.), resistors, multiplexers, registers, capacitors, inductors, diodes, wiring, and so on).

An engine or circuit may be embodied as one or more processing circuits comprising one or more processors communicatively coupled to one or more memory or memory devices. In this regard, the one or more processors may execute instructions stored in the memory or may execute instructions otherwise accessible to the one or more processors. The one or more processors may be constructed in a manner sufficient to perform at least the operations described herein. In some embodiments, the one or more processors may be shared by multiple engines or circuits (e.g., engine A and engine B, or circuit A and circuit B, may comprise or otherwise share the same processor which, in some example embodiments, may execute instructions stored, or otherwise accessed, via different areas of memory).

Alternatively or additionally, the one or more processors may be structured to perform or otherwise execute certain operations independent of one or more co-processors. In other example embodiments, two or more processors may be coupled via a bus to enable independent, parallel, pipelined, or multi-threaded instruction execution. Each processor may be provided as one or more suitable processors, application specific integrated circuits (ASICs), field programmable gate arrays (FPGAs), digital signal processors (DSPs), or other suitable electronic data processing components structured to execute instructions provided by memory. The one or more processors may take the form of a single core processor, multi-core processor (e.g., a dual core processor, triple core processor, quad core processor, etc.), microprocessor, etc. In some embodiments, the one or more processors may be external to the apparatus, for example the one or more processors may be a remote processor (e.g., a cloud based processor). Alternatively or additionally, the one or more processors may be internal and/or local to the apparatus. In this regard, a given engine or circuit or components thereof may be disposed locally (e.g., as part of a local server, a local computing system, etc.) or remotely (e.g., as part of a remote server such as a cloud based server). To that end, engines or circuits as described herein may include components that are distributed across one or more locations.

An example system for providing the overall system or portions of the embodiments described herein might include one or more computers, including a processing unit, a system memory, and a system bus that couples various system components including the system memory to the processing unit. Each memory device may include non-transient volatile storage media, non-volatile storage media, non-transitory storage media (e.g., one or more volatile and/or non-volatile memories), etc. In some embodiments, the non-volatile media may take the form of ROM, flash memory (e.g., flash memory such as NAND, 3D NAND, NOR, 3D NOR, etc.), EEPROM, MRAM, magnetic storage, hard discs, optical discs, etc. In other embodiments, the volatile storage media may take the form of RAM, TRAM, ZRAM, etc. Combinations of the above are also included within the scope of machine-readable media. In this regard, machine-executable instructions comprise, for example, instructions and data which cause a general purpose computer, special purpose computer, or special purpose processing machines to perform a certain function or group of functions. Each respective memory device may be operable to maintain or otherwise store information relating to the operations performed by one or more associated circuits, including processor instructions and related data (e.g., database components, object code components, script components, etc.), in accordance with the example embodiments described herein.

Although the drawings may show and the description may describe a specific order and composition of method steps, the order of such steps may differ from what is depicted and described. For example, two or more steps may be performed concurrently or with partial concurrence. Also, some method steps that are performed as discrete steps may be combined, steps being performed as a combined step may be separated into discrete steps, the sequence of certain processes may be reversed or otherwise varied, and the nature or number of discrete processes may be altered or varied. The order or sequence of any element or apparatus may be varied or substituted according to alternative embodiments. Accordingly, all such modifications are intended to be included within the scope of the present disclosure as defined in the appended claims. Such variation may depend, for example, on the software and hardware systems chosen and on designer choice. All such variations are within the scope of the disclosure. Likewise, software implementations of the described methods could be accomplished with standard programming techniques with rule-based logic and other logic to accomplish the various connection steps, processing steps, comparison steps, and decision steps.

The foregoing description of embodiments has been presented for purposes of illustration and description. It is not intended to be exhaustive or to limit the disclosure to the precise form disclosed, and modifications and variations are possible in light of the above teachings or may be acquired from this disclosure. The embodiments were chosen and described to explain the principals of the disclosure and its practical application to enable one skilled in the art to utilize the various embodiments and with various modifications as are suited to the particular use contemplated. Other substitutions, modifications, changes and omissions may be made in the design, operating conditions, and arrangement of the embodiments without departing from the scope of the present disclosure as expressed in the appended claims.

1. An ultrasound imaging system comprising:
a transducer configured to transmit and receive a first ultrasound signal in a first plane and a second plane, the first plane orthogonal to the second plane, the transducer configured to emit an ultrasound beam; and
a processing circuit having a processor coupled to a memory device storing instructions thereon that, when executed, cause the processing circuit to perform operations comprising:
receiving first image data obtained by an ultrasound probe along the first plane;
receiving second image data obtained by the ultrasound probe along the second plane;
identifying an anatomical feature based on the first image data and the second image data;
determining, based on the first image data and the second image data, an angle between the ultrasound beam and a normal of the anatomical feature;
determining a transmit direction that would adjust the angle between the ultrasound beam and the normal of the anatomical feature to a desired angle; and
at least one of outputting the transmit direction via a user interface of the ultrasound imaging system or automatically aligning a second ultrasound signal in the transmit direction to adjust the angle to the desired angle.

2. The ultrasound imaging system of claim 1, wherein the first plane is an azimuth plane and the second plane is an elevation plane.

3. The ultrasound imaging system of claim 1, wherein the anatomical feature is a pleura.

4. The ultrasound imaging system of claim 3, wherein the desired angle is 0° with respect to the normal of the pleura such that adjusting the ultrasound beam and/or the ultrasound probe to the desired angle adjusts the ultrasound beam to be perpendicular to the pleura.

5. The ultrasound imaging system of claim 1, wherein the operations further comprise causing the transducer to transmit the second ultrasound signal and to receive third image data after automatically aligning the second ultrasound signal to adjust the angle to the desired angle.

6. The ultrasound imaging system of claim 1, wherein the operations further comprise using the user interface to provide real-time feedback to a user to manually adjust an angle of the ultrasound probe such that the transducer can transmit the second ultrasound signal in the desired angle and consequently receive third image data.

7. The ultrasound imaging system of claim 1, wherein determining the transmit direction comprises:
determining a first angle between the ultrasound beam in the first plane and a first normal of the anatomical feature in the first image data; and
determining a second angle between the ultrasound beam in the second plane and a second normal of the anatomical feature in the second image data.

8. The ultrasound imaging system of claim 1, wherein:
the first image data and the second image data comprise pixels; and
identifying the anatomical feature comprises:
training a segmentation model for segmentation of the anatomical feature, the segmentation model configured to output a probability for each pixel being the anatomical feature or not the anatomical feature; and
determining the pixels that classify as the anatomical feature with a threshold such that a binary mask of the anatomical feature is produced.

9. The ultrasound imaging system of claim 8, wherein the anatomical feature is represented as a first vector in the first image data and as a second vector in the second image data, the first vector and the second vector determined by using a method to fit a boundary of the anatomical feature into a best-fit line.

10. The ultrasound imaging system of claim 1, wherein the operations further comprise:
receiving third image data along the first plane and receiving fourth image data along the second plane from the second ultrasound signal responsive to at least one of a user adjusting the ultrasound probe to the transmit direction to transmit the second ultrasound signal or automatically aligning the second ultrasound signal in the transmit direction to transmit the second ultrasound signal;
determining, based on the third image data and the fourth image data, a second angle between the ultrasound beam and a normal of the anatomical feature;
determining a second transmit direction that would adjust the second angle between the ultrasound beam and the normal of the anatomical feature to the desired angle; and
at least one of outputting the second transmit direction via a user interface of the ultrasound imaging system or automatically aligning a third ultrasound signal in the second transmit direction to adjust the second angle to the desired angle.

11. The ultrasound imaging system of claim 1, wherein the transducer is further configured to transmit and receive ultrasound signals in a plurality of planes, the plurality of planes to form a 3D image, and wherein the first plane and the second plane are extracted from the 3D image.

12. An ultrasound imaging system comprising:
a transducer configured to transmit and receive a first ultrasound signal in a first plane and a second plane, the second plane orthogonal to the first plane, the transducer configured to emit an ultrasound beam, the transducer to obtain first image data along the first plane and the transducer to obtain second image data along the second plane;
an image processing circuit configured to identify an anatomical feature based on the first image data and the second image data, wherein an angle between the ultrasound beam and a normal of the anatomical feature is determined;
a control circuit configured to compute, based on the anatomical feature identified in the first image data and the anatomical feature identified in the second image data, a transmit direction that would adjust the angle between the ultrasound beam and the normal of the anatomical feature towards a desired angle; and,
the ultrasound imaging system is configured to at least one of output feedback based on the transmit direction via a user interface of the ultrasound imaging system or automatically align a second ultrasound signal in the transmit direction to adjust the angle to the desired angle.

13. The ultrasound imaging system of claim 12, wherein the anatomical feature is a pleura, and the desired angle is 0° with respect to the normal of the pleura such that adjusting the ultrasound beam to the desired angle adjusts the ultrasound beam to be perpendicular to the pleura.

14. The ultrasound imaging system of claim 12, wherein the control circuit computes the transmit direction with operations comprising:
determining a first angle between the ultrasound beam in the first plane and a first normal of the anatomical feature in the first image data; and
determining a second angle between the ultrasound beam in the second plane and a second normal of the anatomical feature in the second image data.

15. The ultrasound imaging system of claim 12, wherein:
the first image data and the second image data comprise pixels; and
identifying the anatomical feature comprises:
training a segmentation model for segmentation of the anatomical feature, the segmentation model configured to output a probability for each pixel being the anatomical feature or not the anatomical feature; and
determining the pixels that classify as the anatomical feature with a threshold such that a binary mask of the anatomical feature is produced.

16. A method comprising:
receiving, by a processing circuit, first image data obtained by an ultrasound probe along a first plane, the ultrasound probe configured to transmit an ultrasound beam;
receiving, by the processing circuit, second image data obtained by the ultrasound probe along a second plane, the second plane orthogonal to the first plane;
identifying, by the processing circuit, an anatomical feature based on the first image data and the second image data;
determining, by the processing circuit, the first image data, and the second image data, an angle between the ultrasound beam, and a normal of the anatomical feature;
determining, by the processing circuit, a transmit direction that would adjust the angle between the ultrasound beam and the normal of the anatomical feature towards a desired angle; and
at least one of outputting the transmit direction via a user interface or automatically aligning an ultrasound signal in the transmit direction to adjust the angle to the desired angle.

17. The method of claim 16, wherein the anatomical feature is a pleura, and the desired angle is 0° with respect to the normal of the pleura such that adjusting the ultrasound beam and/or the ultrasound probe to the desired angle adjusts the ultrasound beam to be perpendicular to the pleura.

18. The method of claim 16, further comprising providing, by the processing circuit and the user interface, real-time feedback to a user to manually move the ultrasound probe towards the transmit direction such that a transducer can transmit the second ultrasound signal and receive third image data.

19. The method of claim 16, wherein:
the first image data and the second image data comprise pixels; and
identifying the anatomical feature comprises:
training a segmentation model for segmentation of the anatomical feature, the segmentation model configured to output a probability for each pixel being the anatomical feature or not the anatomical feature; and
determining the pixels that classify as the anatomical feature with a threshold such that a binary mask of the anatomical feature is produced.

20. The method of claim 16, wherein determining the transmit direction comprises:
determining a first angle between the ultrasound beam in the first plane and a first normal of the anatomical feature in the first image data; and
determining a second angle between the ultrasound beam in the second plane and a second normal of the anatomical feature in the second image data.
